(19) United States
(12) Patent Application Publication
(10) Pub. No.: US 2016/0338759 A1
(43) Pub. Date: Nov. 24, 2016

(54) NEUTRAL ELECTRODE DEVICE FOR APPLICATION OF RF CURRENT, ELECTROSURGICAL SYSTEM COMPRISING A CORRESPONDING NEUTRAL ELECTRODE DEVICE, AND METHOD FOR PRODUCING A NEUTRAL ELECTRODE DEVICE

(71) Applicant: Erbe Elektromedizin GmbH, Tübingen (DE)

(72) Inventors: Uwe Schnitzler, Tübingen (DE); Peter Selig, Nehren (DE)

(21) Appl. No.: 15/158,184

(22) Filed: May 18, 2016

(30) Foreign Application Priority Data
May 19, 2015 (EP) 15168061.8

(51) Int. Cl. A61B 18/12; A61B 18/10

(52) U.S. Cl. CPC A61B 18/1206; A61B 18/10; A61B 2018/00601

(57) ABSTRACT

The invention relates to a neutral electrode device for use in the application of an RF current to a biological tissue, comprising:
a supporting structure (40) having a first and a second side;
at least one electrode (34, 34′), which is arranged on the first side of the supporting structure (40);
a phase change material (PCM) for absorbing heat, which is arranged on the second side of the supporting structure (40),

wherein the phase change material is formed at least in part as blocks (37, 37′) and the blocks (37, 37′) are arranged on the second side of the supporting structure (40) at least partially distanced from one another in order to form spacing gaps (38, 38′).

### RELATED APPLICATION(S)

This application claims the benefit of European Patent Application No. EP 15168061.8 filed May 19, 2015, the contents of which are incorporated herein by reference as if fully rewritten herein.

### TECHNICAL FIELD

The invention relates to a neutral electrode device having a latent heat store, in particular a phase change material (PCM), a system having a corresponding neutral electrode device, and a method for producing a neutral electrode device.

### BACKGROUND

In radio-frequency surgery (RF surgery), alternating current of high frequency is conducted through the human body in order to purposefully treat or cut tissue. A significant advantage compared with conventional cutting techniques using a scalpel is that, at the same time as the cutting, bleeding can be reduced or even stopped by means of closure of the relevant vessels.

A monopolar technique is often used. Here, one pole of the RF voltage source is connected over the greatest possible area to the patient. The electrode is referred to as a neutral electrode. The other pole (active electrode) is located on a surgical instrument. The current flows from the active electrode to the neutral electrode. The current density is greatest in the immediate vicinity of the active electrode. Here, coagulation or cutting of the tissue takes place.

In the case of neutral electrodes it must be ensured that an excessively high transfer resistance does not occur between the skin and adjacent electrode. High transfer resistance would lead to a significant heating of the biological tissue, and occasionally to burns. Recently, there has been the problem of development of numerous methods in which relatively high RF currents are applied over a longer period of time. The risk of creating burns at the neutral electrode is thus increased. Here, it is also noted that, due to the physical conditions at the edge regions of the neutral electrode, maximum heating occurs there. The risk of burning is therefore particularly high in these edge regions.

In order to avoid undesired damage to the tissue, neutral electrodes having a correspondingly large surface are used, which contribute to reducing the current density in the immediate vicinity of the neutral electrode. In addition, there are monitoring apparatuses, the purpose of which is to identify a partial detachment of the neutral electrode and to respond to this event accordingly.

Current standards stipulate tests that limit a temperature rise at the neutral electrode to a maximum value under application of a certain current over a predefined period of time.

DE 10 2008 046 300 A1 discloses a neutral electrode device which uses phase change material to absorb heat and therefore to absorb thermal peaks. A problem of this neutral electrode device lies in the fact that it must be formed with a relatively small surface area in order to ensure that it securely sits on the tissue.

Reference is also made to U.S. Pat. No. 6,183,855 B1, which generally discloses the use of phase change material in items of clothing.

Proceeding from DE 10 2008 046 300 A1, the object of the present invention is to specify an improved neutral electrode device. In particular, the capability to absorb heat with good contact behaviour (contact between the neutral electrode device and the tissue) is to be improved.

Furthermore, a corresponding electrosurgical system and a method for producing a neutral electrode device are to be specified.

### SUMMARY

In particular, the object is achieved by a neutral electrode device for application of RF current (e.g. between 100 kHz and 1 MHz) to a biological tissue, wherein the device comprises:
A supporting structure having a first and a second side;
At least one electrode, which is arranged on the first side of the supporting structure;
Phase change material for absorbing heat, which is arranged on the second side of the supporting structure,
Wherein the phase change material is formed at least in part as blocks with the blocks being arranged on the second side of the supporting structure at least partially distanced from one another in order to form spacing gaps.

The phase change material is cast into blocks of a matrix for improved flexibility, wherein these are arranged at least in portions at distances from one another. Due to the distances or due to the spacing gaps, a higher flexibility is achieved compared with a neutral electrode device coated in a planar manner. A neutral electrode device that can be attached to patients at surfaces provided accordingly for this purpose, for example at the thigh, is thus possible. These surfaces have quite different shapes and rounded forms in different patients. Due to the distances between the blocks, flexibility is achieved, whereby the neutral electrode device can be attached as intended in practically all patients. The blocks are preferably arranged on a second side of the supporting structure of the neutral electrode device. This is preferably the side of the supporting structure of the neutral electrode device that faces away from the tissue when attached to the patient (tissue-remote side).

The supporting structure may comprise a multiplicity of electrodes. Depending on the definition of the supporting structure, electrodes can also be applied to the first side of the supporting structure. The used electrodes are preferably thin-film electrodes and/or are produced from an aluminium alloy.

In one embodiment the neutral electrode device comprises a conductive substance, in particular from the group of viscoelastic fluids. This may be a hydrogel, which improves the closeness of contact between the at least one electrode and the tissue. The conductive substance is preferably provided on the first—tissue-facing—side of the supporting structure.

At least some of the blocks can be formed as geometric bodies. These are preferably polyhedrons in this case. The geometric bodies can be easily formed and arranged in such a way that they ensure a high flexibility of the neutral electrode device.

The blocks or at least some of the blocks may taper in a direction away from the supporting structure. The distances between the blocks therefore are not necessarily constant. It is possible that the distances reduce in the direction toward the feet of the blocks. In accordance with the invention the blocks may contact one another and may be connected in the region of their feet. The aforementioned spacing gaps are formed in the regions distanced further from the supporting structure.

The widening of the blocks in the direction of the supporting structure has the advantage that energy can be absorbed over the greatest possible area, wherein flexibility is hardly restricted. In one embodiment the spacing gaps delimited by the blocks extend along straight lines. By way of example, an entire network of straight lines or spacing gaps may thus be formed. In one embodiment some of these straight lines contact one another at right angles or are arranged adjacently in parallel.

In one (other) embodiment at least some of the intersecting straight lines, along which the spacing gaps are formed, enclose an angle. The minimal angle measured in each case may extend in a range from 30-90°, preferably 50-90°. In this respect, a regular arrangement of the blocks is provided, which ensures a high flexibility.

The blocks may have different shapes. By way of example, they can be formed as rectangles, structures having trapezoidal cross sections, or in the form of teardrops. The feet of the blocks are preferably connected to the supporting structure. The blocks may have a height of at most 1 cm or 8 mm or 6 mm. They preferably have a maximum height of 4 mm, measured from a plane spanned by the supporting structure, in particular by the second side thereof.

In one exemplary embodiment a block has a maximum volume of 1 cm³, preferably 0.5 cm³.

In one embodiment a multiplicity of the blocks have an identical form. The blocks can be arranged on the supporting structure at least partially at constant distance from one another and/or constant offset from one another.

At least some of the blocks may comprise thermochromic dyes and/or colour pigments (for example 0.1 t4, in particular 0.5 to 2.5, % by weight). It is possible to use thermochromic inks in order to make visible a melting state of the phase change material. In this respect, the phase change material comprising the thermochromic dyes serves not only to absorb heat, but also to display a risk situation or a usage condition (for example 50% of the storage capacity of the latent heat store has been used).

The blocks can be produced completely from a uniform mixture of phase change material. In one embodiment at least some of the blocks are multi-layered, wherein the layers preferably extend parallel to the supporting structure. By way of example a first lower layer can be provided with a first phase change material and a second preferably upper layer can be provided with a phase change material. The first phase change material may differ from the second phase change material in terms of the melting point. By way of example, the melting point of the first phase change material may be 5 and/or 10 and/or 20% higher than the melting point of the second phase change material. It is conceivable to provide further phase change materials or layers of phase change materials, which each differ in terms of the melting point. In a preferred embodiment the blocks are produced from phase change materials in such a way that the melting point decreases continuously or in discrete steps with increasing distance from the supporting structure. By way of example, a lower most layer may melt at approximately 30° C. and layers arranged thereabove may melt at 28° C. and 25° C. This leads to an improved transport of heat away from the electrodes and/or the tissue. Alternatively or additionally, the blocks can be formed in such a way that they have a different thermal conductivity at different positions in the layered structure.

Generally, phase change material of which the melting point lies in a range between 20° and 40°, preferably 25° and 30°, can be used in accordance with the invention.

The object specified in the introduction is also achieved by a system, in particular an electrosurgical system for the coagulation and/or cutting of tissue. This system preferably comprises:
A neutral electrode device;
At least one electrosurgical, in particular monopolar instrument;
An RF generator (e.g. operating frequency between 100 kHz and 1 MHz), which is connected to the neutral electrode device and the at least one instrument.

In one embodiment the neutral electrode device is formed as explained in one of the previous embodiments. Similar advantages to those already mentioned in conjunction with the neutral electrode device are provided.

The object specified in the introduction is also achieved by a method for producing a neutral electrode device. This method may comprise the following steps:
Producing a supporting structure;
Placing a casting mould, which is downwardly open at least in part, onto the supporting structure, wherein the casting mould comprises a multiplicity of segments for producing blocks, which are separated from adjacent segments by partition walls;
Heating a phase change material in such a way that the phase change material is at least partially liquid;
Introducing the heated phase change material into the casting mould;
Removing the casting mould.

In one embodiment the neutral electrode device has some or all of the above-described features.

Advantages similar to those explained in conjunction with the neutral electrode device are also provided in respect of the device.

Due to the use of the downwardly open casting mould, the phase change material can be applied quickly and efficiently.

In one embodiment the production of the supporting structure comprises an application of at least one electrode to a film, in particular a PET film or a PET support. Furthermore, a fibre structure, in particular a non-woven fabric support, can be provided on the side of the film facing away from the electrode. The film and fibre structure can be glued to one another.

The fibre structure is particularly suitable for producing a robust integral bond to the blocks.

The phase change material can be introduced into the casting mould in such a way that an integral bond is produced between the phase change material and the film. Ultimately, some of the phase change material penetrates the fibre structure and contacts the film as said bond is produced. This leads during use to a rapid dissipation of heat when the electrode is hot.

In one embodiment a plurality of phase change materials that differ in particular in terms of melting point are introduced in succession into the costing mould. As a result, the layered structure already explained is formed and the thermal conductivity or transport of heat is improved in turn.

At least one of the phase change materials is processed in a polymer structure. The polymer structure may be sponge-like. The polymer structure may serve for improved processing. Furthermore, it may ensure that the applied blocks retain their shape, even if the phase change material melts partially or wholly during the application. In this respect, the flexibility of the neutral electrode device according to the invention is maintained, even once the phase change material has cured again.

The supporting structure may comprise woven fabric, non-crimp fabric, nonwovens or knitted fabric. Large-mesh woven fabric, non-crimp fabric, nonwovens or knitted fabric are preferably used, which enable an at least partial infiltration and/or penetration of the phase change material in an at least partially liquefied state. This simplifies the production and improves the thermal conductivity of the neutral electrode device. The woven fabric, non-crimp fabric, non-wovens or knitted fabric may for example have a low thread density in the longitudinal and/or transverse direction, for example at most 30 or 20 or 10 threads per centimetre.

Further advantageous embodiments will become clearer from the dependent claims. The invention will be explained hereinafter on the basis of some drawings.

In the drawings:

### BRIEF DESCRIPTION OF THE DRAWINGS

FIG. 1 shows an electrosurgical system comprising a monopolar electrosurgical instrument for cutting and/or coagulating tissue and also comprising a neutral electrode device;

FIG. 2 shows a schematic detailed view of the neutral electrode device according to FIG. 1 having a multiplicity of PCM blocks, which are arranged on a supporting structure;

FIG. 3 shows an exemplary section through the neutral electrode device, in particular a PCM block, of FIG. 2;

FIG. 4 shows a side view of a further neutral electrode device comprising trapezoidal PCM blocks;

FIG. 5 shows a further exemplary embodiment of a neutral electrode device comprising cylindrical PCM blocks;

FIG. 6 shows a section through a further neutral electrode device, wherein the PCM blocks are multi-layered;

FIG. 7 shows a plan view of an exemplary casting mould for producing a neutral electrode device according to FIG. 1.

The same reference numerals will be used in the following description for like and similarly acting parts.

### DETAILED DESCRIPTION

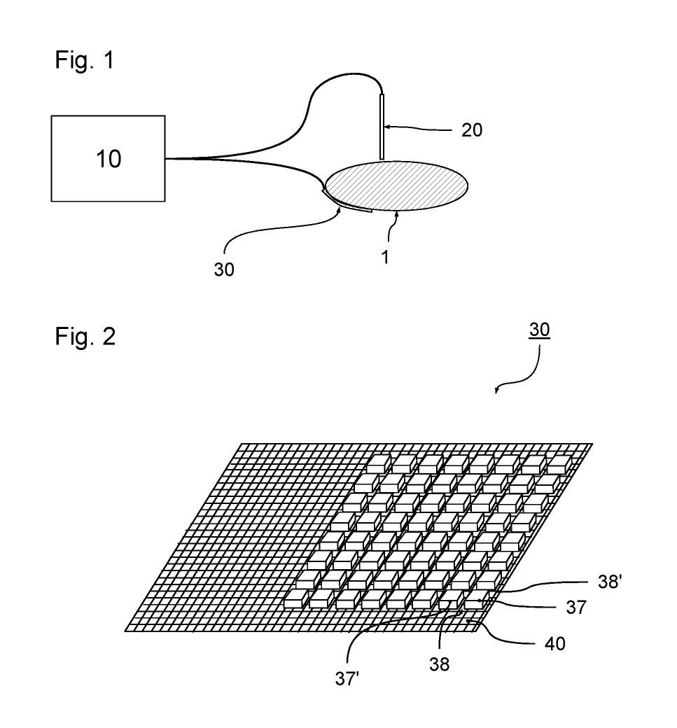

FIG. 1 shows an electrosurgical system. This comprises an RF generator 10, a monopolar instrument 20 and a neutral electrode device 30. With application of an RF current, a voltage (e.g. between 100 V and 5000 V) is applied between the monopolar instrument 20 and the neutral electrode device 30. The RF treatment current (e.g. several Amperes) flows through biological tissue of a body to be treated, in the present case a torso 1. In the immediate vicinity of the monopolar instrument 20, the current density is high, such that the adjacent tissue is coagulated or severed.

In order to avoid burns at the neutral electrode device 30, a latent heat store is provided in accordance with the invention. FIG. 2 shows a detailed view of a corresponding neutral electrode device 30, wherein this comprises a supporting structure 40 and a multiplicity of PCM blocks 37, 37′ arranged on the supporting structure 40.

The PCM blocks 37, 37′ are arranged regularly, specifically in a matrix arrangement. In order to ensure maximum flexibility of the neutral electrode device 30, the PCM blocks 37, 37′ are distanced from one another. There are thus valleys between the individual PCM blocks 37, 37′, which form spacing gaps 38 and 38′ in a longitudinal arrangement and transverse arrangement respectively. In the shown exemplary embodiment the spacing gaps 38, 38′ extend perpendicularly or parallel to one another. They form a grid of spacing gaps 38, 38′.

In the exemplary embodiment the PCM blocks 37, 37′ have a substantially square base area and a height of approximately 3 mm (measured from the surface of the supporting structure 40 to the outer surface of the PCM blocks 37, 37′).

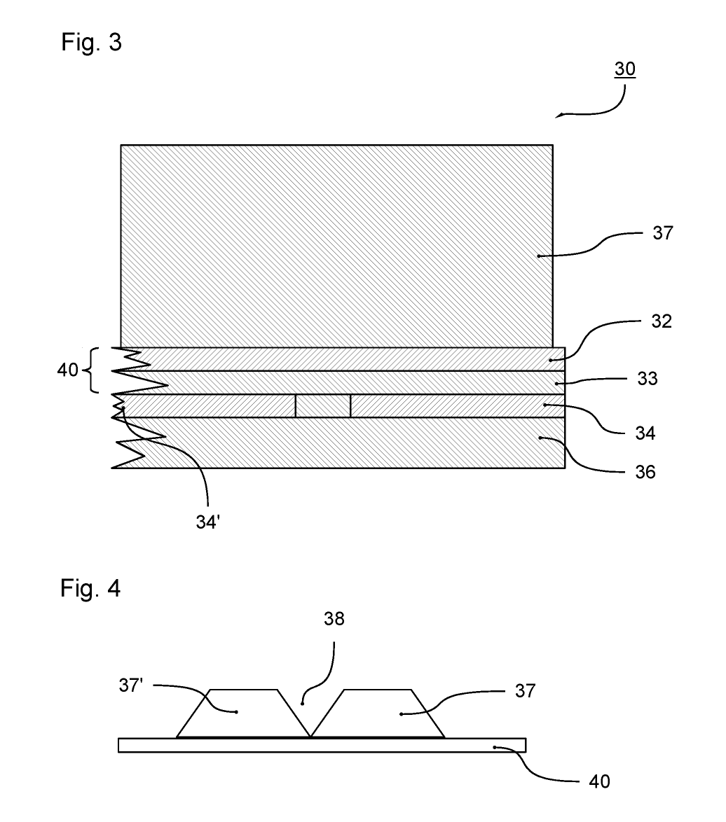

FIG. 3 shows a schematic section through the edge region of the neutral electrode device 30 from FIG. 2. Key components of the neutral electrode device 30 in accordance with the section are the supporting structure 40 with its power (tissue-remote) and its lower (tissue-facing) side and the PCM Block 37. This PCM block 37 is arranged and secured on the upper side of the supporting structure 40. A plurality of electrodes 34, 34′, which are distanced from one another, are located on the lower side of the supporting structure 40. A hydrogel layer 36 covers the electrodes 34, 34′ and in the arranged state forms the direct close contact with the tissue, for example the skin of a patient.

The supporting structure 40 comprises a non-woven fabric support 32 and a PET support 33. In the described exemplary embodiment the PET support 33 is glued to the non-woven fabric support 32. The PET support 33 may have a thickness of 20-80 μm. In the shown exemplary embodiment the thickness thereof amounts to 50 μm. The PET support 33 is used for the mechanical stability of the neutral electrode device 30 and is connected on the lower side to a PET portion of the electrodes 34, 34′ and on the other side to the non-woven fabric support 32. The non-woven fabric support 32 may be a polyester non-woven fabric. The adhesively bonded connection to the PET support 32 can be produced via a biocompatible adhesive.

In the described exemplary embodiment the supporting structure 40 comprises the PET support 33 and the non-woven fabric support 32. In accordance with the invention, the supporting structure 40 can also be defined in such a way that it also comprises the electrodes 34, 34′ and where applicable the layer of hydrogel 36.

The PCM blocks 37, 37′ comprise a sponge-like polymer structure, which is used to provide improved processing and ensures that the applied structure of the PCM blocks 37, 37′ retains its shape even when the PCM melts.

FIG. 4 schematically shows a side view of a further neutral electrode device 30. Here as well, PCM blocks 37, 37′ are arranged on a supporting structure 40. The feet of the blocks 37, 37′ are connected to the supporting structure 40, as is also the case in the previous exemplary embodiment. The PCM blocks 37 taper with increasing distance from the supporting structure 40. In the shown exemplary embodiment the blocks have a trapezoidal shape in their longitudinal section and in their cross section. It is conceivable to form the PCM blocks 37, 37′ in such a way that a corresponding trapezoidal embodiment is provided only in one of the two sections. In accordance with the invention, the feet of the PCM blocks 37, 37′ may contact one another or may be slightly distanced from one another in this exemplary embodiment. The distance between the side walls of the PCM blocks 37, 37′ increases with increasing distance from the supporting structure 40. The spacing gaps 38, 38′ are formed, for example the spacing gap 38 shown in FIG. 4, which extends in the longitudinal direction.

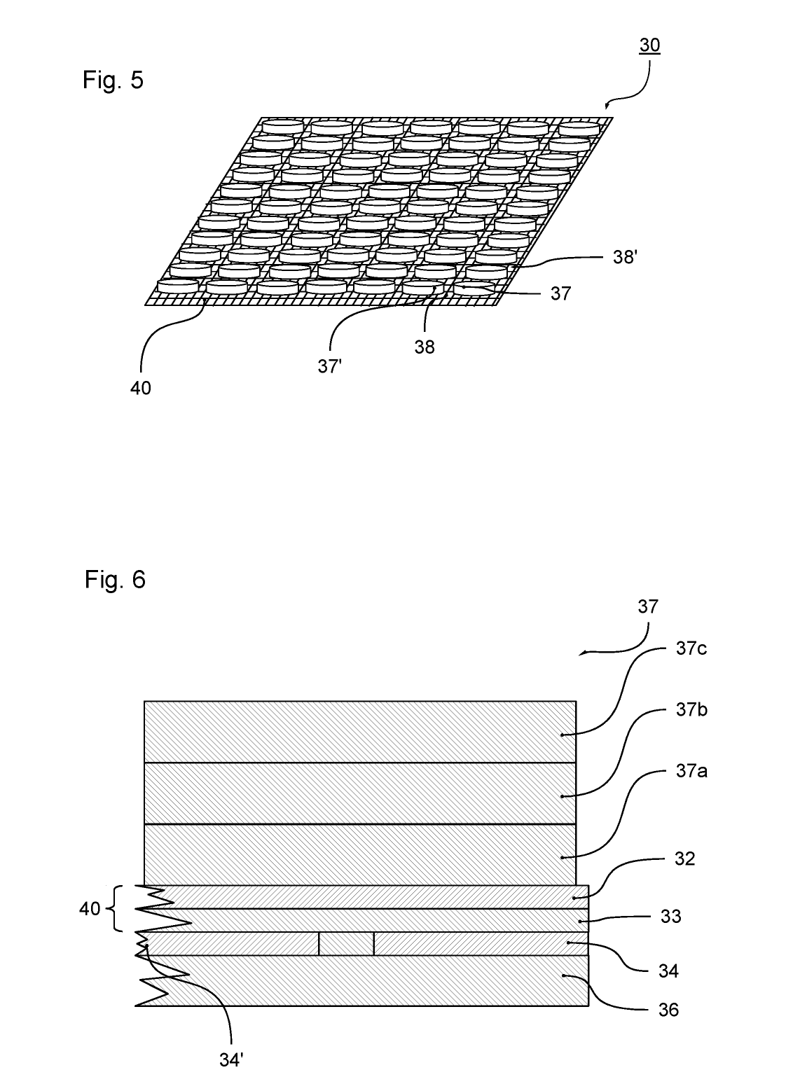

FIG. 5 shows a further embodiment of the neutral electrode device 30 according to the invention. Here, the blocks 37, 37′ are formed as vertical or upright circular cylinders and are also secured to a supporting structure 40, as in the previously described exemplary embodiments. In this exemplary embodiment as well, the arrangement is provided in a sort of matrix. In accordance with the invention, the PCM blocks 37, 37′ may also be arranged offset from one another. By way of example, it is conceivable to modify the exemplary embodiment according to FIG. 5 or according to FIG. 2 in such a way that a second row of PCM blocks is arranged offset by approximately 3 mm from a first row of PCM blocks. In this exemplary embodiment some of the spacing gaps 38, 38′ intersect one another at an obtuse or acute angle.

FIG. 6 corresponds in terms of some features to FIG. 3. However, in the shown exemplary embodiment the PCM block 37 has a multi-layered structure. From bottom (close to the tissue) to top (far from the tissue) the PCM block 37 comprises a first PCM layer 37a, a second PCM layer 37b, and a third PCM layer 37c. In accordance with the invention in the case of this multi-layered structure of the PCM block 37, the PCMs used may each have different melting points. By way of example, the first PCM layer 37a may be configured in such a way that this melts at 30° C. The second PCM layer 37b and the third PCM layer 37c can be configured in such a way that they melt at 28° C. and 25° C. respectively. The third PCM layer thus melts first and removes heat from the underlying layer. This leads to an improved transport of heat away from the tissue against which the neutral electrode device is applied. In accordance with the invention, more or fewer layers can be provided. The layer transition between the individual PCM layers 37a, 37b, 37c also does not have to be discrete and/or planar, as shown in FIG. 6. The individual PCM layers 37a, 37b, 37c may mix with one another at their boundary regions and/or may form boundary layers which have curvatures and craters. In the described exemplary embodiment a key aspect is that there are at least two points within the PCM block 37 at which the used PCM has a different melting point. Here, the melting point of the point further removed from the supporting structure 40 is preferably lower than the other.

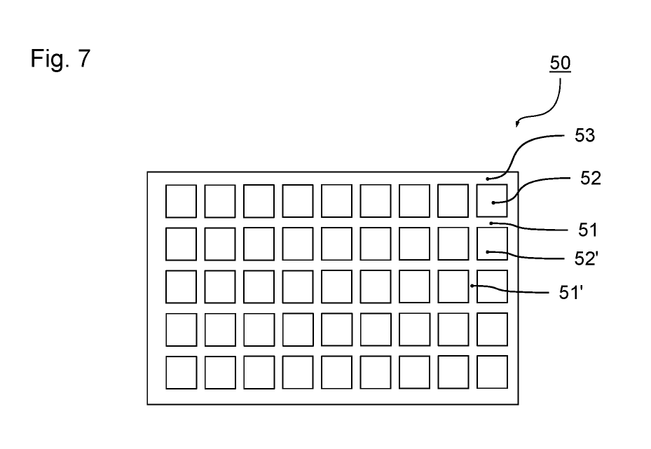

FIG. 7 schematically shows a plan view of a casting mould 50, with which the neutral electrode device 30 according to FIG. 2 can be produced, for example. This has a substantially rectangular frame 53, within which a multiplicity of partition walls 51, 51′ are arranged in transverse and longitudinal direction. The partition walls 51, 51′ delimit upwardly and downwardly open segments 52, 52′. A method according to the invention for producing a neutral electrode device may comprise the following steps:

Producing a supporting structure 40;
Placing the casting mould 52 onto the supporting structure 40;
Heating phase change material in such a way that this liquefies at least in part;
Pouring the phase change material into the casting mould 50 in such a way that the segments 52, 52′ are filled at least in part, and
Removing the casting mould 50.

The removal preferably takes place once the blocks 37, 37′ have cured at least in part. Where necessary, different phase change materials, for example for producing the described layered structure, can be introduced in succession. It is also conceivable to use a mixing ratio of the PCM during the introduction process that changes over time, such that, for example, the melting point of the PCM decreases with increasing distance from the supporting structure 40.

Further optional details for the possible production of a supporting structure 40 according to the invention with PCM will be described hereinafter.

The individual specified and described exemplary embodiments can be combined with one another arbitrarily in accordance with the invention. By way of example, a multi-layered structure of the PCM blocks 37, 37′ may also be selected in the exemplary embodiment according to FIG. 4. The casting mould 50 according to FIG. 7 can also be modified in such a way that the neutral electrode device 30 according to FIG. 5 can be produced herewith. Furthermore, an offset or irregular arrangement of the PCM blocks 37 is also conceivable with the neutral electrode device 30 by way of example. The PCM blocks 37 of a neutral electric device 30 may also comprise different shapes (squares, cylinders, cubes, trapezoids, pyramids, etc.).

### LIST OF REFERENCE SIGNS

1 torso
10 RF generator
20 monopolar instrument
30 neutral electrode device
32 non-woven fabric support
33 PET support
34, 34′ electrodes
36 hydrogel
37, 37′ PCM block
37, 37b, 37c PCM layer
38, 38′ spacing gap
40 supporting structure
50 casting mould
51, 51′ partition wall
52, 52′ segment
53 frame

1. An apparatus comprising a neutral electrode device for application of an RF current to a biological tissue, the neutral electrode device comprising:
A supporting structure (40) having a first and a second side;
At least one electrode (34, 34′), which is arranged on the first side of the supporting structure (40);
Phase change material (PCM) for absorbing heat, which is arranged on the second side of the supporting structure (40),
wherein the phase change material is formed at least in part as blocks (37, 37′) and the blocks (37, 37′) are arranged on the second side of the supporting structure (40) at least partially distanced from one another in order to form spacing gaps (38, 38′).

2. The neutral electrode device according to claim 1, further comprising a multiplicity of electrodes (34, 34′) produced from an aluminium alloy.

3. The neutral electrode device according to claim 1, further comprising a conductive substance disposed on the at least one electrode, wherein the conductive substance comprises a viscoelastic fluid.

4. The neutral electrode device according to claim 1, wherein the blocks (37, 37′) are formed as geometric bodies.

5. The neutral electrode device according to claim 1, wherein the blocks (37, 37′) taper in a direction away from the supporting structure.

6. The neutral electrode device according to claim 1, wherein the spacing gaps (38, 38′) delimited by the blocks (37, 37′) extend over an entirety of the supporting structure (40) along straight lines.

7. The neutral electrode device according to claim 1, wherein a multiplicity of the blocks (37, 37′) have an identical form and are arranged on the supporting structure (40) at constant distance from one another and/or constant offset from one another.

8. The neutral electrode device according to claim 1, wherein at least some of the blocks (37, 37′) comprise thermochromic dyes and/or colour pigments.

9. The neutral electrode device according to claim 1, wherein at least some of the blocks (37, 37′) comprise a first, lower, layer (37a) having a first phase change material and a second, upper, layer (37c) having a second phase change material, wherein the first phase change material has a melting point that is significantly higher, by at least 5%, than a melting point of the second phase change material.

10. The apparatus of claim 1 further comprising an electrosurgical system for the coagulation and/or cutting of tissue, comprising:
the neutral electrode device (30)
At least one electrosurgical monopolar instrument (20);
An RF generator, which is connected to the neutral electrode device (30) and the at least one instrument (20).

11. A method for producing a neutral electrode device, said method comprising:
Producing a supporting structure (40);
Placing a casting mould (50), which is downwardly open at least in part, onto the supporting structure (40), wherein the casting mould (50) comprises a multiplicity of segments (52, 52′) for producing blocks (37, 37′), which are separated from adjacent segments (52, 52′) by partition walls (51, 51′);
Heating a phase change material (PCM) in such a way that the phase change material is at least partially liquid;
Introducing the heated phase change material into the casting mould (50);
Removing the casting mould (50).

12. The method according to claim 11, wherein the production of the supporting structure (40) comprises an application of at least one electrode (34, 34′) to a film, and the application of a fibre structure to a side of the film facing away from the electrode (34, 34′).

13. The method according to claim 12, wherein the fibre structure is arranged and the phase change material is introduced into the casting mould (50) in such a way that an integral bond is produced between the phase change materials and the film.

14. The method according to claim 11, further comprising introducing in succession into the casting mould a plurality of different phase change materials having different melting points.

15. The method according to claim 11, further comprising processing at least one phase change material in a sponge-like polymer structure.
